(19) United States
(12) Patent Application Publication
(10) Pub. No.: US 2020/0323740 A1
(43) Pub. Date: Oct. 15, 2020

(54) PROTECTIVE PAD FOR A NIPPLE

(71) Applicant: Katina Johnson, San Antonio, TX (US)

(72) Inventor: Katina Johnson, San Antonio, TX (US)

(21) Appl. No.: 16/382,436

(22) Filed: Apr. 12, 2019

(51) Int. Cl. A61J 13/00; A61F 13/14

(52) U.S. Cl. CPC A61J 13/00; A61F 13/141

(57) ABSTRACT

A protective pad to fit around a portion of at least one of a breast, a nipple, and an areola of a user, the protective pad including a main body to contact at least a portion of the breast of the user, and a protective dome disposed on a center portion of the main body to protect at least one of the breast, the nipple, and the areola.

### BACKGROUND

### 1. Field

The present general inventive concept relates generally to a pad, and particularly, to a protective pad for a nipple.

### 2. Description of the Related Art

For a majority of female patients undergoing a mastectomy, at least a portion of a breast, a nipple and an areola are removed. Many women choose to undergo nipple reconstruction surgery. While the breast and the nipple recover, an area around a portion of skin of the nipple is tender.

Additionally, after childbirth, many women experience heightened sensitivity and discomfort around the nipple. As such, a new mother will seek a means to cover the breast and/or the nipple from touch and/or other abrasive contact.

Currently, nipple dressings do not have durability, flexibility, and/or allow airflow within the nipple dressings.

Therefore, there is a need for a protective pad to protect the nipple that is durable and flexible, while it recovers.

### SUMMARY

The present general inventive concept provides a protective pad for a nipple.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a protective pad to fit around a portion of at least one of a breast, a nipple, and an areola of a user, the protective pad including a main body to contact at least a portion of the breast of the user, a protective dome disposed on a center portion of the main body to protect at least one of the breast, the nipple, and the areola.

The main body may include an adhesive layer disposed on at least a portion of the main body to attach the main body to the portion of the breast of the user.

The protective pad may further include an adhesive covering layer removably connected to the adhesive layer to protect the adhesive layer from wear prior to attaching the main body to the portion of the breast of the user.

The protective dome may include at least one breathing aperture disposed on a portion of the protective dome to permit air to flow in and out of the protective dome.

The protective pad may further include an electronic information transceiver to store at least one of a name, a date, a time, contact information, and a type of medical procedure performed on the user.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a top isometric view of a protective pad, according to an exemplary embodiment of the present general inventive concept; and

FIG. 2 illustrates a bottom isometric view of the protective pad, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

List of Components

Protective Pad 100
Main Body 110
First Side 110a
Second Side 110b
Third Side 110c
Fourth Side 110d
Fifth Side 110e
Aperture 111
Adhesive Layer 112
Protective Dome 120
At Least One Breathing Aperture 121
Adhesive Covering Layer 130
Electronic Information Transceiver 140

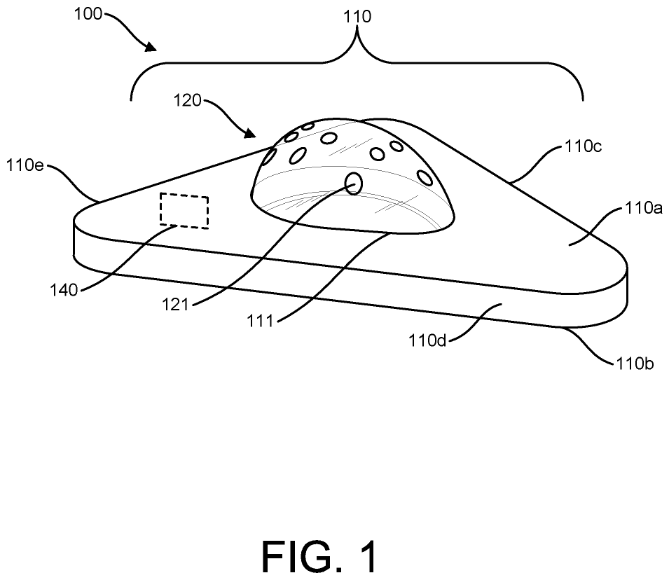

FIG. 1 illustrates a top isometric view of a protective pad 100, according to an exemplary embodiment of the present general inventive concept.

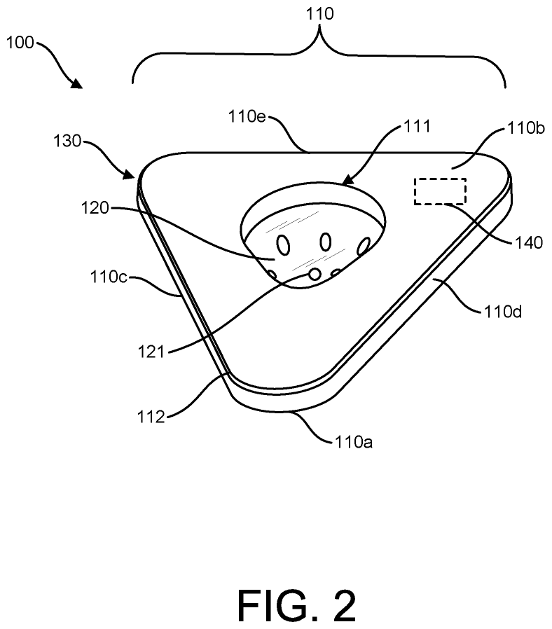

FIG. 2 illustrates a bottom isometric view of the protective pad 100, according to an exemplary embodiment of the present general inventive concept.

The protective pad 100 may be constructed from at least one of metal, plastic, wood, cotton, wool, polyester, polyethylene, polyurethane, and rubber, etc., but is not limited thereto. Additionally, the protective pad 100 may be waterproof. Furthermore, the protective pad 100 may be highly durable and flexible to provide long-lasting support.

Referring to FIGS. 1 and 2, the protective pad 100 may include a main body 110, a protective dome 120, an adhesive covering layer 130, and an electronic information transceiver 140, but is not limited thereto.

Referring to FIGS. 1 and 2, the main body 110 is illustrated to have a triangular prism shape. However, the main body 110 may be a square prism, a rectangular prism, a circular prism, a conical prism, a pentagonal prism, a hexagonal prism, an octagonal prism, or any other shape known to one of ordinary skill in the art, but is not limited thereto.

Referring to FIGS. 1 and 2, the main body 110 may include a first side 110a, a second side 110b, a third side 110c, a fourth side 110d, a fifth side 110e, an aperture 111, and an adhesive layer 112, but is not limited thereto.

The main body 110 may be of any predetermined size to accommodate at least one of a breast, a nipple, and an areola of a woman's body, but is not limited thereto. Specifically, the main body 110 may be constructed to fit around at least a portion of the breast, the nipple, and/or the areola. Alternatively, the main body 110 may be constructed to be a one size fits all, but is not limited thereto. As such, the main body 110 may cushion any contact by an object against the breast, the nipple, and/or the areola.

The aperture 111 may be disposed on at least a center portion of the main body 110. The aperture 111 may be of any predetermined size to fit around a circumference of the breast, the nipple, and/or the areola of the woman's body.

The protective dome 120 may include at least one breathing aperture 121, but is not limited thereto.

The protective dome 120 may be disposed within at least a portion of the aperture 111. Moreover, the protective dome 120 may have a diameter substantially similar to a diameter of the aperture 111, such that the protective dome 120 may snugly fit within the aperture 111. The protective dome 120 may extend a predetermined distance away from the first side 110a of the main body 110 to accommodate the breast, the nipple, and/or the areola. In other words, at least one of the breast, the nipple, and the areola may be inserted into the protective dome 120, such that the breast, the nipple, and/or the areola may be protected therein.

The at least one breathing aperture 121 may be disposed on at least a portion of the protective dome 120. Additionally, the at least one breathing aperture 121 may permit air to flow in and/or out of the protective dome 120, such that air may contact the breast, the nipple, and/or the areola. As such, free-flowing air may facilitate a healing process of the breast, the nipple, and/or the areola after surgery. As such, the free-flowing air may prevent an infection from developing on the breast, the nipple, and/or the areola under the protective dome 120.

The adhesive layer 112 may include a glue, a tape, a sticker, and/or any combination thereof, but is not limited thereto.

The main body 110 may be highly resistant to falling off the woman's body due to the adhesive layer 112. The adhesive layer 112 may be disposed on at least a portion of the second side 110b. Alternatively, the adhesive layer 112 may be disposed on the first side 110a, the third side 110c, the fourth side 110d, and/or the fifth side 110e, but is not limited thereto.

Referring to FIG. 2, the adhesive covering layer 130 may be removably connected to the adhesive layer 112. Moreover, the adhesive covering layer 130 may protect the adhesive layer 112 from wear prior to attaching the main body 110 to the woman's body. The adhesive covering layer 130 may be pushed and/or pulled away from the adhesive layer 112 to remove the adhesive covering layer 130. Subsequently, a user may grasp at least one of the first side 110a, the third side 110c, the fourth side 110d, and the fifth side 110e to direct the main body 110 toward the woman's body, such that the adhesive layer 112 is affixed and/or adhered to the woman's body, such that the breast, the nipple, and/or the areola may protrude into the protective dome 120. Furthermore, the main body 110 may not be removed without a substantial application of force to remove the main body 110 from the woman's body. In other words, the main body 110 may not fall off the woman's body unless the user attempts to remove the protective pad 100. As such, the main body 110 may be removably connected to the woman's body.

The electronic information transceiver 140 may include at least one of a barcode, a quick response (QR) code, a radio-frequency identification (RFID) tag, a near field communication (NFC) tag, and a storage unit, but is not limited thereto. The electronic information transceiver 140 may store at least one of a name, a date, a time, contact information, and a type of medical procedure performed on the woman, but is not limited thereto. The electronic information transceiver 140 may be updated by a medical provider and may assist the medical provider in keeping tracking of information regarding the woman wearing the protective pad 100.

As such, the protective pad 100 may be an ideal product to help women that have suffered from breast cancer, are a new mother, or are recovering from breast and nipple reconstruction surgery.

Therefore, a protective pad 100 may fit around a portion of at least one of a breast, a nipple, and an areola of the user, the protective pad 100 may include the main body 110 to contact at least a portion of the breast of the user, the protective dome 120 disposed on a center portion of the main body 110 to protect at least one of the breast, the nipple, and the areola. The main body 110 may include the adhesive layer 112 disposed on at least a portion of the main body 110 to attach the main body 110 to the portion of the breast of the user. The protective pad 100 may further include the adhesive covering layer 130 removably connected to the adhesive layer 112 to protect the adhesive layer 112 from wear prior to attaching the main body 110 to the portion of the breast of the user. The protective dome 120 may include the at least one breathing aperture 121 disposed on a portion of the protective dome 120 to permit air to flow in and out of the protective dome 120. The protective pad 120 may further include the electronic information transceiver 140 to store at least one of a name, a date, a time, contact information, and a type of medical procedure performed on the user.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A protective pad to fit around a portion of at least one of a breast, a nipple, and an areola of a user, the protective pad comprising:
a main body to contact at least a portion of the breast of the user; and
a protective dome disposed on a center portion of the main body to protect at least one of the breast, the nipple, and the areola.

2. The protective pad of claim 1, wherein the main body comprises:
an adhesive layer disposed on at least a portion of the main body to attach the main body to the portion of the breast of the user.

3. The protective pad of claim 2, further comprising:
an adhesive covering layer removably connected to the adhesive layer to protect the adhesive layer from wear prior to attaching the main body to the portion of the breast of the user.

4. The protective pad of claim 1, wherein the protective dome comprises:
at least one breathing aperture disposed on a portion of the protective dome to permit air to flow in and out of the protective dome.

5. The protective pad of claim 1, further comprising:
an electronic information transceiver to store at least one of a name, a date, a time, contact information, and a type of medical procedure performed on the user.
